(19) United States
(12) Patent Application Publication
(10) Pub. No.: US 2013/0336909 A1
(43) Pub. Date: Dec. 19, 2013

(54) PERSONAL CARE AND HEALTH CARE COMPOSITIONS

(71) Applicants: Jean-Luc Garaud, Comines (FR); Bethany K. Johnson, Midland, MI (US); Stephanie Postiaux, Ressaix (BE); Xavier Jean-Paul Thomas, Famars (FR); Stephane Ugazio, Soignies (BE); Isabelle Van Reeth, Shanghai (CN)

(72) Inventors: Jean-Luc Garaud, Comines (FR); Bethany K. Johnson, Midland, MI (US); Stephanie Postiaux, Ressaix (BE); Xavier Jean-Paul Thomas, Famars (FR); Stephane Ugazio, Soignies (BE); Isabelle Van Reeth, Shanghai (CN)

(73) Assignee: Dow Corning Corporation, Midland, MI (US)

(21) Appl. No.: 13/994,849

(22) PCT Filed: Jan. 9, 2012
(86) PCT No.: PCT/EP12/50259

§ 371 Date: Sep. 5, 2013

Related U.S. Application Data
(60) Provisional application No. 61/431,165, filed on Jan. 10, 2011.

(51) Int. Cl. A61K 8/895; A61Q 1/12; A61Q 1/06; A61Q 3/02; A61Q 5/10; A61Q 17/04

(52) U.S. Cl. CPC A61K 8/895; A61Q 5/10; A61Q 17/04; A61Q 1/06; A61Q 3/02; A61Q 1/12
USPC 424/59; 514/772.3; 424/70.12; 424/64; 424/61

(57) ABSTRACT

The present invention relates to health care and personal care compositions comprising a silicone acrylate hybrid composition and at least one health care and/or personal care ingredient. Such compositions tend to provide desirable characteristics for the care and conditioning of hair and skin.

### BACKGROUND OF THE INVENTION

Disclosed herein are compositions comprising a silicone acrylate hybrid composition and at least one ingredient that is useful in personal care compositions.

Silicones are present in many hair care products to enhance the shine and healthy appearance of the hair and are also present in skin care products to enhance the smooth feel of the skin.

Cosmetic formulations benefit from film formers for long lasting, substantivity, tunable occlusivity.

The silicone acrylate hybrid composition disclosed herein provides long lasting to color cosmetics and color protection for hair.

### BRIEF SUMMARY OF THE INVENTION

Disclosed herein are compositions comprising a silicone acrylate hybrid composition and at least one ingredient that is useful in personal care compositions. Such compositions tend to provide desirable characteristics for the care and conditioning of hair and skin such as long lasting, hair color resistance, water proofness.

### DETAILED DESCRIPTION OF THE INVENTION

The silicone acrylate hybrid composition useful herein is described in patent application PCT/US2007/013321 filed on Jun. 6, 2007, the disclosure of which is hereby incorporated by reference.

In particular, the silicone acrylate hybrid composition is the reaction product of (I) a silicon-containing pressure sensitive adhesive that contains acrylate or methacrylate functionality , (II) an ethylenically unsaturated monomer, and (III) an initiator. As used herein, the term silicone acrylate denotes a polymerized hybrid species that includes silicone-based sub-species and acrylate-based sub-species that have been polymerized together.

The silicon-containing pressure sensitive adhesive (I) comprises acrylate or methacrylate functionality. The silicon-containing pressure sensitive adhesive (I) may include only acrylate functionality, only methacrylate functionality, or both acrylate functionality and methacrylate functionality. The silicon-containing pressure sensitive adhesive is present in the hybrid composition in an amount of from 5 to 95, alternatively 25 to 75, parts by weight based on 100 parts by weight of the hybrid composition.

The silicon-containing pressure sensitive adhesive (I) is typically produced by the condensation reaction product of (i) a pressure sensitive adhesive and (ii) a silicon-containing capping agent wherein the capping agent provides the acrylate or methacrylate functionality to the silicon-containing pressure sensitive adhesive (I).

The pressure sensitive adhesive (i) comprises the condensation reaction product of (a) a silicone resin and (b) a polydiorganosiloxane. Although not required, the pressure sensitive adhesive (i) may comprise a catalytic amount of a condensation catalyst.

There is a wide array of silicone resins (a) and polydiorganosiloxanes (b) that are suitable to make up the pressure sensitive adhesive (i). Suitable silicone resins (a) and polydiorganosiloxanes (b) include, but are not limited to, those disclosed and described in U.S. Pat. No. 6,337,086 to Kanios et al., the disclosure of which is incorporated by reference herein in its entirety.

A typical silicone resin (a) comprises triorganosiloxy units of the formula R³₃SiO_{1/2} and tetrafunctional siloxy units of the formula SiO_{4/2} in a ratio of about 0.6 to 0.9 triorganosiloxy units for each tetrafunctional siloxy unit, wherein each R³ independently denotes a monovalent hydrocarbon radical having from 1 to 6 carbon atoms such as methyl, ethyl, propyl, isopropyl, hexyl, hexenyl, cyclohexyl, vinyl, allyl, propenyl and phenyl.

There may also be a few mole percent of R₃₂Siₒ units present in the silicone resin (a) provided that the presence of such units does not cause the ultimate product to lose its ability to function as a PSA.

Silicone resin (a) may be prepared according to Daudt et al., U.S. Pat. No. 2,676,182 (issued Apr. 20, 1954 and hereby incorporated by reference) whereby a silica hydrosol is treated at a low pH with a source of R³₃SiO_{1/2} units such as a hexaorganodisiloxane such as Me₃SiOSiMe₃, ViMe₂SiOSiMe₂Vi or MeViPhSiOSiPhViMe or triorganosilane such as Me₃SiCl, Me₂ViSiCI or MeViPhSiCl.

The silicone resins (a) contain silicon-bonded hydroxyl radicals in amounts which typically range from about 1 to 4 weight percent of silicon-bonded hydroxyl radicals. There should be at least some and alternatively at least 0.5% silicon-bonded hydroxyl content to enable the polydiorganosiloxane (b) to copolymerize with the silicone resin (a). The silicon-bonded hydroxyl radicals may also react with the endblocking agent being added to chemically treat the pressure sensitive adhesive (i).

The silicone resins (a) are generally benzene-soluble resinous materials which are typically solids at room temperature and are prepared as, and usually, but not necessarily used as, a solution in an organic solvent or cosmetic solvent. Typical organic solvents used to dissolve silicone resins (a) include benzene, toluene, xylene, methylene chloride, perchloroethylene, naphtha mineral spirits and mixtures of these. Typical cosmetic solvents used to dissolve silicone resins (a) include cyclomethicone, dimethicone, ethanol, isopropyl alcohol, mineral oil, sunflower oil, caprylic/capric triglyceride.

A typical polydiorganosiloxane (b) comprises AR³SiO units with terminal TR³ASiO_{1/2} units, where each A radical is independently selected from R³ or halohydro-carbon radicals having from 1 to 6 carbon atoms such a chloromethyl, chloropropyl, 1-chloro-2-methylpropyl, 3,3,3-trifluoropropyl and F₃C(CH₂)₅ radicals, each T radical is independently selected from the group consisting of R³, OH, H or OR⁴, and each R⁴ is independently an alkyl radical having from 1 to 4 carbon atoms such as methyl, ethyl, n-propyl, and isobutyl radicals.

Polydiorganosiloxane (b) has a viscosity of from 100 centipoise to 30,000,000 centipoise at 25° C. Polydiorganosiloxanes (b) having a viscosity of from about 100 to 100,000 centipoise at 25° C. range from fluids to somewhat viscous polymers. These polydiorganosiloxanes (b) may be prereacted with silicone resin (a) prior to condensation in the presence of an endblocking agent to improve the tack and adhesion properties of the resulting pressure sensitive adhesive (i) as will be further described. Polydiorganosiloxanes (b) having viscosities in excess of 100,000 centipoise may typically be subjected to the condensation/endblocking step without prereaction. Polydiorganosiloxanes (b) having viscosities in excess of 1,000,000 centipoise are highly viscous products often referred to as gums and the viscosity is often expressed in terms of a Williams Plasticity value (polydimethylsiloxane gums of about 10,000,000 centipoise viscosity typically have a Williams Plasticity Value of about 1.27 mm (50 mils) or more at 25° C.).

Polydiorganosiloxane (b) typically has the formula AR³SiO such as Me₂SiO units, PhMeSiO units, MeViSiO units, Ph₂SiO units, methylethylsiloxy units, 3,3,3-trifluoropropyl units and 1-chloro, 2-methylpropyl units and the like.

Alternatively, the AR³SiO units are selected from the group consisting of R³₂SiOR³R⁴SiO units, Ph₂SiO units and combinations of both. At least 50 mole percent of the R⁴ radicals present in the polydiorganosiloxane (b) are methyl radicals and no more than 50 mole percent of the total moles of AR³SiO units present in each polydiorganosiloxane (b) are Ph₂SiO units. Alternatively, no more than 10 mole percent of the AR³SiO units present in each polydiorganosiloxane (b) are MeR³SiO units and the remaining AR³SiO units present in each polydiorganosiloxane (b) are Me₂SiO units. Alternatively, substantially all of the AR³SiO units are Me₂SiO units.

The H, OH and OR⁴ in the terminal units of polydiorganosiloxane (b) provide a site for reaction with the acrylate or methacrylate functional silicon-containing capping agent (ii) and also provide a site for condensation with other such radicals on polydiorganosiloxanes (b) or with the silicon-bonded hydroxyl groups present in silicone resin (a). Use of polydiorganosiloxanes (b) where T is OH is appropriate because the polydiorganosiloxane (b) may then readily copolymerize with the silicone resin (a). When an appropriate catalyst such as HCl, which is generated when chlorosilanes are used, or ammonia, which is generated when organosilazanes are used as endblocking agents, then triorganosiloxy (e.g., R³₃SiO_{1/2} such as (CH₃)₃SiO_{1/2} or CH₂CH(CH₃)₂ SiO_{1/2}) unit terminated polydiorganosiloxanes (b) may be employed because some of the triorganosiloxy units may be cleaved when the condensation reaction is conducted with heating. The cleavage exposes a silicon-bonded hydroxyl radical which may then condense with silicon-bonded hydroxyl radicals in the silicone resin (a), with endblocking triorganosilyl units or with other polydiorganosiloxanes (b) containing H, OH or OR⁴ radicals or silicon-bonded hydroxyl radicals exposed by cleavage reactions. Mixtures of polydiorganosiloxanes (b) containing different substituent radicals may also be used.

When pressure sensitive adhesives (i) are to be cured by peroxide or through aliphatically unsaturated radicals present in silicone resin (a) or polydiorganosiloxane (b), if silicone resin (a) contains aliphatically unsaturated radicals, then polydiorganosiloxane (b) should be free of such radicals and vice-versa. If both components contain aliphatically unsaturated radicals, curing through such radicals may result in products which do not act as pressure sensitive adhesives.

The pressure sensitive adhesives (i) are made using from 30 to 80, alternatively 40 to 75, parts by weight of silicone resins (a) and from 20 to 70, alternatively 25 to 60, parts by weight of polydiorganosiloxane (b). Although not required, the pressure sensitive adhesive (i) may comprise a catalytic amount of a condensation catalyst.

The pressure sensitive adhesive (i), comprising silicon bonded hydroxyl groups (i.e., silanols), and the silicon-containing capping agent (ii) are condensed to produce the silicon-containing pressure sensitive adhesive (I). Once the silicon-containing capping agent (ii) reacts with the pressure sensitive adhesive (i), the concentration of silanols in the silicon-containing pressure sensitive adhesive (I) is typically from 5,000 to 15,000, more typically from 8,000 to 13,000 ppm.

The pressure sensitive adhesive (i) is present in the silicon-containing pressure sensitive adhesive (I) in an amount of from 85.0 to 99.9, alternatively 90.0 to 99.8, alternatively 50 to 65, alternatively 60 parts by weight based on weight % solids of the silicon-containing pressure sensitive adhesive (I), and the silicon-containing capping agent (ii) is present in the silicon-containing pressure sensitive adhesive (I) in an amount of from 0.1 to 15, alternatively 0.2 to 10 parts by weight based on weight % solids of the silicon-containing pressure sensitive adhesive (I).

The silicon-containing capping agent (ii) may be introduced to react with the pressure sensitive adhesive (i) after the pressure sensitive adhesive (i) has already been formed, i.e., after the silicone resin (a) and the polydiorganosiloxane (b) which make up the pressure sensitive adhesive (i) have condensation reacted.

Alternatively, the silicon-containing capping agent (ii) may be reacted in situ with the silicone resin (a) and the polydiorganosiloxane (b) such that the silicon-containing capping agent (ii) is present as the silicone resin (a) and the polydiorganosiloxane (b) are reacting to form the pressure sensitive adhesive (i). That is, in this in situ scenario, the silicon-containing capping agent (ii) is introduced either prior to or during the reaction of the silicone resin (a) and the polydiorganosiloxane (b) to form the pressure sensitive adhesive (i).

The silicon-containing capping agent (ii) is selected from the group of acrylate functional silanes, acrylate functional silazanes, acrylate functional disilazanes, acrylate functional disiloxanes, methacrylate functional silanes, methacrylate functional silazanes, methacrylate functional disilazanes, methacrylate functional disiloxanes, and combinations thereof.

The silicon-containing capping agent (ii) may be described to be of the general formula (XYR₂Si)₂D wherein X is a monovalent radical of the general formula A′E- where E is —O— or —NH— and A′ is an acryl group or a methacryl group, Y is a divalent alkylene radical having from 1 to 6 carbon atoms, R is a methyl or a phenyl radical, and D is a divalent or a trivalent organic hydrolyzable radical. Alternatively, D is —O— or —NH—. This particular silicon-containing capping agent (ii) may be selected from the group of Bis(3-methacryloxypropyl)tetramethyldisilazane, Bis(3-acryloxypropyl)tetramethyldisilazane, Bis(3-methacryloxypropyl)tetramethyldisiloxane, Bis(3-acryloxypropyl) tetramethyldisiloxane, and combinations thereof.

Alternatively, the silicon-containing capping agent (ii) may be described to be of the general formula XYR′_{b}SiZ_{3-b} wherein R′ is a methyl or a phenyl radical, Z is a monovalent hydrolyzable organic radical or a halogen, and b is 0, 1, or 2. Alternatively, the monovalent hydrolyzable organic radical is of the general formula R″O— where R″ is an alkylene radical.

This particular silicon-containing capping agent (ii) may be selected from the group of 3-methacryloxypropyldimethylchlorosilane, 3-methacryloxypropyldichlorosilane, 3-methacryloxypropyltrichlorosilane, 3-methacryloxypropyldimethylmethoxysilane, 3-methacryloxypropylmethyldimethoxysilane, 3-methacryloxypropyltrimethoxysilane, 3-methacryloxypropyldimethylethoxysilane, 3-methacryloxypropylmethyldiethoxysilane, 3-methacryloxypropyltriethosysilane, (methacryloxymethyl)dimethylmethoxysilane, (methacryloxymethyl)methyldimethoxysilane, (methacryloxymethyl)trimethoxysilane, (methacryloxymethyl)dimethylethoxysilane, (methacryloxymethyl)methyldiethoxysilane, methacryloxymethyltriethoxysilane, methacryloxypropyltriisopropoxysilane, 3-methacryloxypropyldimethylsilazane, 3-acryloxypropyldimethylchlorosilane, 3-acryloxypropyldichlorosilane, 3-acryloxypropyltrichlorosilane, 3-acryloxypropyldimethylmethoxysilane, 3-acryloxypropylmethyldimethoxysilane, 3-acryloxypropyltrimethoxysilane, 3-acryloxypropyldimethylsilazane, and combinations thereof.

A second silicon-containing capping agent (iii) may be used in conjunction with the silicon-containing capping agent (ii). This second silicon-containing capping agent (iii) is distinguishable from the silicon-containing capping agent (ii) in that the second silicon-containing capping agent (iii) is free of acrylate and methacrylate functionality and is capable of generating an endblocking triorganosilyl unit. If included, the second silicon-containing capping agent (iii) contributes to the reaction forming the silicon-containing pressure sensitive adhesive (I), along with the silicon-containing capping agent (ii) and the pressure sensitive adhesive (i). Suitable second silicon-containing capping agents (iii) include, but are not limited to, those described in U.S. Pat. No. 6,337,086 to Kanios et al., the disclosure of which has already been incorporated by reference for its teaching of these silicon-containing capping agents (iii).

The ethylenically unsaturated monomer (II) is a reactant that, along with the silicon-containing pressure sensitive adhesive (I) and the initiator (III), reacts to form the silicone acrylate hybrid composition of the present invention.

The ethylenically unsaturated monomer (II) is present in the silicone acrylate hybrid composition in an amount of from 5 to 95, alternatively from 25 to 75, parts by weight based on 100 parts by weight of the silicone acrylate hybrid composition.

The ethylenically unsaturated monomer (II) may be any monomer having at least one carbon-carbon double bond. The ethylenically unsaturated monomer (II) used in the present invention may be a compound selected from the group of aliphatic acrylates, aliphatic methacrylates, cycloaliphatic acrylates, cycloaliphatic methacrylates, and combinations thereof. It is to be understood that each of the compounds, the aliphatic acrylates, the aliphatic methacrylates, the cycloaliphatic acrylates, and the cycloaliphatic methacrylates, include an alkyl radical which may include up to 20 carbon atoms.

The aliphatic acrylates that may be selected as one of the ethylenically unsaturated monomers (II) are selected from the group consisting of methyl acrylate, ethyl acrylate, propyl acrylate, n-butyl acrylate, iso-butyl acrylate, tert-butyl acrylate, hexyl acrylate, 2-ethylhexyl acrylate, iso-octyl acrylate, iso-nonyl acrylate, iso-pentyl acrylate, tridecyl acrylate, stearyl acrylate, lauryl acrylate, and mixtures thereof. The aliphatic methacrylates that may be selected as one of the ethylenically unsaturated monomers are selected from the group consisting of methyl methacrylate, ethyl methacrylate, propyl methacrylate, n-butyl methacrylate, iso-butyl methacrylate, tert-butyl methacrylate, hexyl methacrylate, 2-ethylhexyl methacrylate, iso-octyl methacrylate, iso-nonyl methacrylate, iso-pentyl methacrylate, tridecyl methacrylate, stearyl methacrylate, lauryl methacrylate, and mixtures thereof. The cycloaliphatic acrylate that may be selected as one of the ethylenically unsaturated monomers is cyclohexyl acrylate, and the cycloaliphatic methacrylate that may be selected as one of the ethylenically unsaturated monomers is cyclohexyl methacrylate. Styrene may also be selected.

Certain other monomers, described herein as polar monomers, may be used as the ethylenically unsaturated monomer (II) and may include supplemental functionality such as hydroxyl functionality. A polar monomer as used herein is an acrylic or methacrylic monomer having at least one polar group such as hydroxyl, alkoxy, amino, and alkenyl heterocycles. Examples of these polar monomers that are useful in the present invention include, but are not limited to, hydrophilic ethylenically unsaturated monomers of an amphoteric, anionic, cationic or anionic nature which are polymerizable by radical polymerization. More specific examples of these polar monomers include, but are not limited to, acrylic acid, methacrylic acid, itaconic acid, vinyl acetic acid, hydroxy ethyl acrylate, hydroxy ethyl methacrylate, hydroxy propyl acrylate, hydroxy propyl methacrylate, methoxyethyl acrylate, methoxyethyl methacrylate, aminoethyl acrylate, aminoethyl methacrylate, 2-N,N,N-trimethylammonium ethyl acrylate, 2-N,N,N-trimethylammonium ethyl methacrylate, acrylonitrile, methacrylonitrile, N,N-dimethylacrylamide, N-t-butylacrylamide, acrylamide, N-vinyl pyrrolidone, 2-acrylamido-2-methyl propane sulphonic acid, or salts thereof and the like.

The ethylenically unsaturated monomer (II) and the silicon-containing pressure sensitive adhesive (I) are polymerized in the presence of the initiator (III). The polymerization of the ethylenically unsaturated monomer (II) and the silicon-containing pressure sensitive adhesive (I) in the presence of the initiator (III) may be conducted at a temperature of from 50 to 100° C., alternatively of from 65 to 90° C.

The method of making the silicone acrylate hybrid composition may be employed in a batch process, semi-continuous process, or continuous process.

Although not required, the silicon-containing pressure sensitive adhesive (I), the ethylenically unsaturated monomer (II), and the initiator (III) may be mixed to form a pre-reaction mixture prior to the step of polymerizing and this pre-reaction mixture may be combined with a solvent prior to the step of polymerization. If these optional steps are conducted, then the polymerization obviously occurs with the components in the pre-reaction mixture after the pre-reaction mixture has been combined with the solvent.

It is to be understood that there are many different initiation mechanisms contemplated for use in the present invention to initiate the polymerization of the silicon-containing pressure sensitive adhesive (I) and the ethylenically unsaturated monomer (II). However, the typical initiator (III) is that known throughout the art as a free radical initiator.

Free radical initiators include peroxides, azo compounds, redoxinitiators, and photo-initiators. The typical free radical initiators for application in the present invention are selected from the group of peroxides, azo compounds, and combinations thereof.

The initiator (III) is present in the silicone acrylate hybrid composition in an amount of from 0.005 to 3, alternatively from 0.01 to 2, parts by weight based on 100 parts by weight of the hybrid composition.

During the polymerization of the ethylenically unsaturated monomer (II) and the silicon-containing pressure sensitive adhesive (I), the silicone to acrylic ratio may be sufficiently controlled and optimized as desired. Controlling the silicone to acrylic ratio is desirable because the silicone acrylate hybrid composition may be optimized dependent on the end application for the silicone acrylate hybrid composition. An illustrative example of a mechanism is the rate controlled addition of the ethylenically unsaturated monomer or monomers (II) to the silicon-containing pressure sensitive adhesive (I). In certain applications, it may be desirable to have the silicone-based sub-species, or the overall silicone content, to exceed the acrylate-based sub-species, or the overall acrylic content. In other applications, it may be desirable for the opposite to be true. Independent of the end application, it is typical that the silicon-containing pressure sensitive adhesive (I) is present in the silicone acrylate hybrid composition in an amount of from 5 to 95, alternatively from 25 to 75, parts by weight based on 100 parts by weight of the silicone acrylate hybrid composition.

A solvent may be used during the polymerization to make the silicone acrylate hybrid composition to decrease the viscosity of the reaction mixture which allows for adequate mixing and heat transfer. The solvent may be any suitable material which is inert to the reaction ingredients and does not interfere with the reaction itself. Suitable solvents include, but are not limited to, aliphatic hydrocarbons such as hexane and heptane; alcohols such as methanol, ethanol and butanol; ketones such as acetone, methyl ethyl ketone and methyl isobutyl ketone; esters such as ethyl acetate, n-butyl acetate and i-butyl acetate; low viscosity silicone oils with linear, cyclic or branched structures which have a boiling point below 250° C. and a viscosity below 100 centipoises such as octamethylcyclotetrasiloxane, decamethylcyclopentasiloxane and hexamethyldisiloxane; and mixtures of two or more of the above mentioned solvents. If utilized, the amount of solvent is alternatively present in an amount of from 30 to 95, alternatively 40 to 70, parts by weight based on the total amount of the reactants and solvent.

The polymerization may occur in emulsion and the resulting silicone acrylate hybrid composition forming a silicone-in-water emulsion. The silicon-containing pressure sensitive adhesive (I), the ethylenically unsaturated monomer (II), and the initiator (III) may be mixed together and then emulsified to form a pre-reaction mixture prior to the step of polymerizing;

they may be combined with a solvent prior to the step of emulsification. If these optional steps are conducted, then the polymerization occurs with the components in the pre-reaction mixture after the pre-reaction mixture has been emulsified in water. The emulsion may be obtained by various emulsification techniques known by persons of ordinary skill in the art such as but not limited to mechanical emulsion, twin screw extrusion, emulsion stabilized with emulsifier, surfactant or thickener, and pickering emulsion.

For cases where the molecular weight of the polymerization is to be controlled or limited, a chain transfer agent may be used. Chain transfer agents are known in the art and may include mercaptans, such as 1-butanethiol and dodecanethiol. If utilized, the amount of the chain transfer agent is alternatively from about 0 to 0.5 parts by weight per 100 parts by weight of the silicone acrylate hybrid composition.

Once the silicone acrylate hybrid composition has been prepared, it is combined with at least one ingredient that is useful in personal care compositions.

Personal care ingredients are those ingredients that are useful in the field of personal care. The ingredients involved are in particular the ingredients used for the care of the skin, such as moisturizing, cleaning, deep cleansing, tightening, toning, skin lightening, protective (in particular protective against ultraviolet radiation or other aggressive factors, such as the cold and atmospheric pollution), anti-aging (in particular anti-wrinkle and/or firming) and slimming cares; for the care of the hair, such as conditioning, colouring, straightening, volumizing, shine; for the care of nails such as hardening and colouring.

Personal care ingredients include emollients, waxes, moisturizers, surface active materials such as surfactants or detergents or emulsifiers, sebum absorbents or sebum control agents, vegetable or botanical extracts, pigments, colorants, thickeners, silicone conditioning agents, cationic conditioning agents, UV absorbers and sunscreen agents, preservatives, anti-dandruff agents, vitamins, proteins and amino-acid and their derivatives, hair dyes, nail care ingredients, fragrances, pH controlling agents, cosmetic biocides, antioxidants, oxidizing agents, reducing agents, skin bleaching agents, skin protectants and a cosmetically acceptable medium, such as water or mixtures thereof.

Additional ingredients that may be used in the personal care compositions include antiperspirants, fatty alcohols, colour care additives, pearlising agents, electrolytes, chelating agents, film formers, styling agents, ceramides, suspending agents and others.

Examples of emollients include volatile or non-volatile silicone oils; silicone resins such as polypropylsilsesquioxane and phenyl trimethicone; silicone elastomers such as dimethicone crosspolymer; alkylmethylsiloxanes such as C30-45 Alkyl Methicone; volatile or non-volatile hydrocarbon compounds, such as squalene, paraffin oils, petrolatum oils and naphthalene oils; hydrogenated or partially hydrogenated polyisobutene; isoeicosane; squalane; isoparaffin; isododecane; isodecane or isohexa- decane; branched C8-Ci6 esters; isohexyl neopentanoate; ester oils such as isononyl isononanoate, cetostearyl octanoate, isopropyl myristate, palmitate derivatives, stearates derivatives, isostearyl isostearate and the heptanoates, octanoates, decanoates or ricinoleates of alcohols or of polyalcohols, or mixtures thereof; hydrocarbon oils of plant origin, such as wheatgerm, sunflower, grapeseed, castor, shea, avocado, olive, soybean, sweet almond, palm, rapeseed, cotton seed, hazelnut, macadamia, jojoba, blackcurrant, evening primrose; or triglycerides of caprylic/capric acids; higher fatty acids, such as oleic acid, linoleic acid or linolenic acid.

Example of waxes include hydrocarbon waxes such as beeswax, lanolin wax, rice wax, carnauba wax, candelilla wax, microcrystalline waxes, paraffins, ozokerite, polyethylene waxes.

Examples of moisturizers include lower molecular weight aliphatic diols such as propylene glycol and butylene glycol; polyols such as glycerine and sorbitol; and polyoxyethylene polymers such as polyethylene glycol 200; hyaluronic acid and its derivatives.

Examples of surface active materials or emulsifiers may be anionic, cationic or non ionic, and include organomodified silicones such as dimethicone copolyol; oxyethylenated and/or oxypropylenated ethers of glycerol; oxyethylenated and/or oxypropylenated ethers of fatty alcohols such as ceteareth-30, C12-15 pareth-7; fatty acid esters of polyethylene glycol such as PEG-50 stearate, PEG-40 monostearate; saccharide esters and ethers, such as sucrose stearate, sucrose cocoate and sorbitan stearate, and mixtures thereof; phosphoric esters and salts thereof, such as DEA oleth-10 phosphate; sulphosuccinates such as disodium PEG-5 citrate lauryl sulphosuccinate and disodium ricinoleamido MEA sulphosuccinate; alkyl ether sulphates, such as sodium lauryl ether sulphate; isethionates; betaine derivatives.

Examples of sebum absorbents or sebum control agents include silica silylate, silica dimethyl silylate, dimethicone/vinyl dimethicone crosspolymer, polymethyl methacrylate, cross-linked methylmethacrylate and aluminum starch octenylsuccinate.

Examples of vegetable or botanical extracts are derived from plants (herbs, roots, flowers, fruits, or seeds) in oil or water soluble form, such as coconut, green tea, white tea, black tea, horsetail, sunflower, wheat germ, olive, grape, pomegranate, apricot, carrot, tomato, tobacco, bean, potato, actzuki bean, catechu, orange, cucumber, avocado, watermelon, banana, lemon or palm. Examples of herbal extracts include dill, horseradish, oats, neem, beet, broccoli, tea, pumpkin, soybean, barley, walnut, flax, ginseng, poppy, avocado, pea or sesame.

Examples of pigments and colorants include surface treated or untreated iron oxides, surface treated or untreated titanium dioxide, surface treated or untreated mica, silver oxide, silicates, chromium oxides, carotenoids, carbon black, chlorophyllin derivatives and yellow ocher.

Examples of thickeners include acrylamide copolymers, acrylate copolymers and salts thereof, xanthan gum and derivatives, cellulose gum and cellulose derivatives, carbomer, cassia gum, guar gum, cocamide derivatives, alkyl alcohols, gelatin, PEG-derivatives.

Examples of silicone conditioning agents for hair include silicone oils; silicone gums and mixtures or emulsions thereof; organomodified silicone oils, such as amodimethicone, aminopropyl phenyl trimethicone, phenyl trimethicone, trimethyl pentaphenyl trisiloxane, silicone quaternium-16/glycidoxy dimethicone crosspolymer, silicone quaternium-16 and mixtures or emulsions thereof.

Examples of cationic conditioning agents include guar derivatives, quaternary nitrogen derivatives of cellulose ethers; homopolymers of dimethyldiallyl ammonium chloride; copolymers of acrylamide and dimethyldiallyl ammonium chloride; homopolymers or copolymers derived from acrylic acid or methacrylic acid which contain cationic nitrogen functional groups attached to the polymer by ester or amide linkages; polycondensation products of N,N′-bis-(2,3-epoxypropyl)-piperazine or piperazine-bis-acrylamide and piperazine; and copolymers of vinylpyrrolidone and acrylic acid esters with quaternary nitrogen functionality. Specific materials include the various polyquats Polyquaternium-7, Polyquaternium-8, Polyquaternium-10, Polyquaternium-11, and Polyquaternium-23. Other categories of conditioners include cationic surfactants such as cetyl trimethylammonium chloride, cetyl trimethylammonium bromide, and stearyltrimethylammonium chloride.

UV absorbers and sunscreen agents include those which absorb ultraviolet light between about 290-320 nanometers (the UV-B region) and those which absorb ultraviolet light in the range of 320-400 nanometers (the UV-A region).

Some examples of sunscreen agents are aminobenzoic acid, cinoxate, diethanolamine methoxycinnamate, digalloyl trioleate, dioxybenzone, ethyl 4-[bis(Hydroxypropyl)] aminobenzoate, glyceryl aminobenzoate, homosalate, lawsone with dihydroxyacetone, menthyl anthranilate, octocrylene, ethyl hexyl methoxycinnamate, octyl salicylate, oxybenzone, padimate O, phenylbenzimidazole sulfonic acid, red petrolatum, sulisobenzone, titanium dioxide, and trolamine salicylate.

Some examples of UV absorbers are acetaminosalol, allatoin PABA, benzalphthalide, benzophenone, benzophenone 1-12, 3-benzylidene camphor, benzylidenecamphor hydrolyzed collagen sulfonamide, benzylidene camphor sulfonic Acid, benzyl salicylate, bornelone, bumetriozole, butyl Methoxydibenzoylmethane, butyl PABA, ceria/silica, ceria/silica talc, cinoxate, DEA-methoxycinnamate, dibenzoxazol naphthalene, di-t-butyl hydroxybenzylidene camphor, digalloyl trioleate, diisopropyl methyl cinnamate, dimethyl PABA ethyl cetearyldimonium tosylate, dioctyl butamido triazone, diphenyl carbomethoxy acetoxy naphthopyran, disodium bisethylphenyl tiamminotriazine stilbenedisulfonate, disodium distyrylbiphenyl triaminotriazine stilbenedisulfonate, disodium distyrylbiphenyl disulfonate, drometrizole, drometrizole trisiloxane, ethyl dihydroxypropyl PABA, ethyl diisopropylcinnamate, ethyl methoxycinnamate, ethyl PABA, ethyl urocanate, etrocrylene ferulic acid, glyceryl octanoate dimethoxycinnamate, glyceryl PABA, glycol salicylate, homosalate, isoamyl p-methoxycinnamate, isopropyl benzyl salicylate, isopropyl dibenzolylmethane, isopropyl methoxycinnamate, menthyl anthranilate, menthyl salicylate, 4-methylbenzylidene, camphor, octocrylene, octrizole, octyl dimethyl PABA, ethyl hexyl methoxycinnamate, octyl salicylate, octyl triazone, PABA, PEG-25 PABA, pentyl dimethyl PABA, phenylbenzimidazole sulfonic acid, polyacrylamidomethyl benzylidene camphor, potassium methoxycinnamate, potassium phenylbenzimidazole sulfonate, red petrolatum, sodium phenylbenzimidazole sulfonate, sodium urocanate, TEA-phenylbenzimidazole sulfonate, TEA-salicylate, terephthalylidene dicamphor sulfonic acid, titanium dioxide, tri PABA panthenol, urocanic acid, and VA/crotonates/methacryloxybenzophenone-1 copolymer.

Example of preservatives and cosmetic biocides include paraben derivatives, hydantoin derivatives, chlorhexidine and its derivatives, imidazolidinyl urea, phenoxyethanol, silver derivatives, salicylate derivatives, triclosan, zinc pyrithione and mixtures thereof.

Examples of antidandruff agents include pyridinethione salts, selenium compounds such as selenium disulfide, and soluble antidandruff agents.

Examples of vitamins include a variety of different organic compounds such as alcohols, acids, sterols, and quinones. They may be classified into two solubility groups:

lipid-soluble vitamins and water-soluble vitamins. Lipid-soluble vitamins that have utility in personal care formulations include retinol (vitamin A), ergocalciferol (vitamin D2), cholecalciferol (vitamin D3), phytonadione (vitamin K1), and tocopherol (vitamin E). Water-soluble vitamins that have utility in personal care formulations include ascorbic acid (vitamin C), thiamin (vitamin B1) niacin (nicotinic acid), niacinamide (vitamin B3), riboflavin (vitamin

B2), pantothenic acid (vitamin B5), biotin, folic acid, pyridoxine (vitamin B6), and cyanocobalamin (vitamin B12). Additional examples of vitamins include derivatives of vitamins such as retinyl palmitate (vitamin A palmitate), retinyl acetate (vitamin A acetate), retinyl linoleate (vitamin A linoleate), and retinyl propionate (vitamin A propionate), tocopheryl acetate (vitamin E acetate), tocopheryl linoleate (vitamin E linoleate), tocopheryl succinate (vitamin E succinate), tocophereth-5, tocophereth-10, tocophereth-12, tocophereth-18, tocophereth-50 (ethoxylated vitamin E derivatives), PPG-2 tocophereth-5, PPG-5 tocophereth-2, PPG-10 tocophereth-30, PPG-20 tocophereth-50, PPG-30 tocophereth-70, PPG-70 tocophereth-100 (propoxylated and ethoxylated vitamin E derivatives), sodium tocopheryl phosphate, ascorbyl palmitate, ascorbyl dipalmitate, ascorbyl glucoside, ascorbyl tetraisopalmitate, tetrahexadecyl ascorbate, ascorbyl tocopheryl maleate, potassium ascorbyl tocopheryl phosphate or tocopheryl nicotinate.

Proteins or amino-acids and their derivatives include those extracted from wheat, soy, rice, corn, keratin, elastin or silk. Most proteins are in the hydrolyzed form and they may also be quaternized.

Examples of hair dyes include 1-acetoxy-2-methylnaphthalene; acid dyes; 5-amino-4-chloro-o-cresol; 5-amino-2,6-dimethoxy-3-hydroxypyridine; 3-amino-2,6-dimethylphenol; 2-amino-5-ethylphenol HCl; 5-amino-4-fluoro-2-methylphenol sulfate; 2-amino-4-hydroxyethylaminoanisole; 2-amino-4-hydroxyethylaminoanisole sulfate; 2-amino-5-nitrophenol; 4-amino-2-nitrophenol; 4-amino-3-nitrophenol; 2-amino-4-nitrophenol sulfate; m-aminophenol HCl; p-aminophenol HCl; m-aminophenol; o-aminophenol; 4,6-bis(2-hydroxyethoxy)-m-phenylenediamine HCl; 2,6-bis(2-hydroxyethoxy)-3,5-pyridinediamine HCl; 2-chloro-6-ethylamino-4-nitrophenol; 2-chloro-5-nitro-N-hydroxyethyl p-phenylenediamine; 2-chloro-p-phenylenediamine; 3,4-diaminobenzoic acid; 4,5-diamino-1-((4-chlorophenyl)methyl)-1H-pyrazole-sulfate; 2,3-diaminodihydropyrazolo pyrazolone dimethosulfonate; 2,6-diaminopyridine; 2,6-diamino-3-((pyridin-3-yl)azo)pyridine; dihydroxyindole; dihydroxyindoline; N,N-dimethyl-p-phenylenediamine; 2,6-dimethyl-p-phenylenediamine; N,N-dimethyl-p-phenylenediamine sulfate; direct dyes; 4-ethoxy-m-phenylenediamine sulfate; 3-ethylamino-p-cresol sulfate; N-ethyl-3-nitro PABA; gluconamidopropyl aminopropyl dimethicone; Haematoxylon brasiletto wood extract; HC dyes; Lawsonia inermis (Henna) extract; hydroxyethyl-3,4-methylenedioxyaniline HCl; hydroxyethyl-2-nitro-p-toluidine; hydroxyethyl-p-phenylenediamine sulfate; 2-hydroxyethyl picramic acid; hydroxypyridinone; hydroxysuccinimidyl C21-22 isoalkyl acidate; isatin; Isatis tinctoria leaf powder; 2-methoxymethyl-p-phenylenediamine sulfate; 2-methoxy-p-phenylenediamine sulfate ; 6-methoxy-2,3-pyridinediamine HCl; 4-methylbenzyl 4,5-diamino pyrazole sulfate; 2,2′-methylenebis 4-aminophenol; 2,2′-methylenebis-4-aminophenol HCl; 3,4-methylenedioxyaniline; 2-methylresorcinol; methylrosanilinium chloride; 1,5-naphthalenediol; 1,7-naphthalenediol; 3-nitro-p-Cresol; 2-nitro-5-glyceryl methylaniline; 4-nitroguaiacol; 3-nitro-p-hydroxyethylaminophenol; 2-nitro-N-hydroxyethyl-p-anisidine; nitrophenol; 4-nitrophenyl aminoethylurea; 4-nitro-o-phenylenediamine dihydrochloride; 2-nitro-p-phenylenediamine dihydrochloride; 4-nitro-o-phenylenediamine HCl; 4-nitro-m-phenylenediamine; 4-nitro-o-phenylenediamine; 2-nitro-p-phenylenediamine; 4-nitro-m-phenylenediamine sulfate; 4-nitro-o-phenylenediamine sulfate; 2-nitro-p-phenylenediamine sulfate; 6-nitro-2,5-pyridinediamine; 6-nitro-o-toluidine; PEG-3 2,2′-di-p-phenylenediamine; p-phenylenediamine HCl; p-phenylenediamine sulfate; phenyl methyl pyrazolone; N-phenyl-p-phenylenediamine HCl; pigment blue 15:1; pigment violet 23; pigment yellow 13; pyrocatechol; pyrogallol; resorcinol; sodium picramate; sodium sulfanilate; solvent yellow 85; solvent yellow 172; tetraaminopyrimidine sulfate; tetrabromophenol blue; 2,5,6-triamino-4-pyrimidinol sulfate; 1,2,4-trihydroxybenzene

Example of nail care ingredients include butyl acetate; ethyl acetate; nitrocellulose; acetyl tributyl citrate; isopropyl alcohol; adipic acid/neopentyl glycol/trimelitic anhydride copolymer; stearalkonium bentonite; acrylates copolymer; calcium pantothenate; Cetraria islandica extract; Chondrus crispus; styrene/acrylates copolymer; trimethylpentanediyl dibenzoate-1; polyvinyl butyral; N-butyl alcohol; propylene glycol; butylene glycol; mica; silica; tin oxide; calcium borosilicate; synthetic fluorphlogopite; polyethylene terephtalate; sorbitan laurate derivatives; talc; jojoba extract; diamond powder; isobutylphenoxy epoxy resin; silk powder.

Examples of fragrances or perfume include hexyl cinnamic aldehyde; anisaldehyde; methyl-2-n-hexyl-3-oxo-cyclopentane carboxylate; dodecalactone gamma; methylphenylcarbinyl acetate; 4-acetyl-6-tert-butyl-1,1 -dimethyl indane; patchouli; olibanum resinoid; labdanum; vetivert; copaiba balsam; fir balsam; 4-(4-hydroxy-4-methyl pentyl)-3-cyclohexene-1-carboxaldehyde; methyl anthranilate; geraniol; geranyl acetate; linalool; citronellol; terpinyl acetate; benzyl salicylate; 2-methyl-3-(p-isopropylphenyl)-propanal; phenoxyethyl isobutyrate; cedryl acetal; aubepine; musk fragrances; macrocyclic ketones; macrolactone musk fragrances; ethylene brassylate.

Examples of pH controlling agents include any water soluble acid such as a carboxylic acid or a mineral acid such as hydrochloric acid, sulphuric acid, and phosphoric acid, monocarboxylic acid such as acetic acid and lactic acid, and polycarboxylic acids such as succinic acid, adipic acid, and citric acid.

Some examples of cosmetic biocides are aluminium phenolsulfonate, ammonium phenolsulfonate, bakuchiol, benzalkonium bromide, benzalkonium cetyl phosphate, benzalkonium chloride, benzalkonium saccharinate, benzethonium chloride, potassium phenoxide, benzoxiquine, benzoxonium chloride, bispyrithione, boric acid, bromochlorophene, camphor benzalkonium methosulfate, captan, cetalkonium chloride, cetearalkonium bromide, cetethyldimonium bromide, cetrimonium bromide, cetrimonium chloride, cetrimonium methosulfate, cetrimonium saccharinate, cetrimonium tosylate, cetylpyridinium chloride, chloramine T, chlorhexidine, chlorhexidine diacetate, chlorhexidine digluconate, chlorhexidine dihydrochloride, p-chloro-m-cresol, chlorophene, p-chlorophenol, chlorothymol, chloroxylenol, chlorphenesin, ciclopirox olamine, climbazole, cloflucarban, clotrimazole, coal tar, colloidal sulfur, o-cymen-5-ol, dequalinium acetate, dequalinium chloride, dibromopropamidine diisethionate, dichlorobenzyl alcohol, dichlorophene, dichlorophenyl imidazoldioxolan, dichloro-m-xylenol, diiodomethyltolylsulfone, dimethylol ethylene thiourea, diphenylmethyl piperazinylbenzimidazole, domiphen bromide, 7-ethylbicyclooxazolidine, fluorosalan, formaldehyde, glutaral, hexachlorophene, hexamidine, hexamidine diisethionate, hexamidine diparaben, hexamidine paraben, hexetidine, hydrogen peroxide, hydroxymethyl dioxoazabicyclooctane, ichthammol, isopropyl cresol, lapyrium chloride, lauralkonium bromide, lauralkonium chloride, laurtrimonium bromide, laurtrimonium chloride, laurtrimonium trichlorophenoxide, lauryl isoquinolinium bromide, lauryl isoquinolinium saccharinate, laurylpyridinium chloride, mercuric oxide, methenamine, methenammonium chloride, methylbenzethonium chloride, myristalkonium chloride, myristalkonium saccharinate, myrtrimonium bromide, nonoxynol-9 iodine, nonoxynol-12 iodine, olealkonium chloride, oxyquinoline, oxyquinoline benzoate, oxyquinoline sulfate, PEG-2 coco-benzonium chloride, PEG-10 coco-benzonium chloride, PEG-6 undecylenate, PEG-8 undecylenate, phenol, o-phenylphenol, phenyl salicylate, piroctone olamine, sulfosuccinylundecylenate, potassium o-phenylphenate, potassium salicylate, potassium troclosene, propionic acid, PVP-iodine, quaternium-8, quaternium-14, quaternium-24, sodium phenolsulfonate, sodium phenoxide, sodium o-phenylphenate, sodium shale oil sulfonate, sodium usnate, thiabendazole, 2,2′-thiobis(4-chlorophenol), thiram, triacetin, triclocarban, triclosan, trioctyldodecyl borate, undecylenamidopropylamine oxide, undecyleneth-6, undecylenic acid, zinc acetate, zinc aspartate, zinc borate, zinc chloride, zinc citrate, zinc cysteinate, zinc dibutyldithiocarbamate, zinc gluconate, zinc glutamate, zinc lactate, zinc phenolsulfonate, zinc pyrithione, zinc sulfate, and zinc undecylenate.

Some examples of antioxidants are acetyl cysteine, arbutin, ascorbic acid, ascorbic acid polypeptide, ascorbyl dipalmitate, ascorbyl methylsilanol pectinate, ascorbyl palmitate, ascorbyl stearate, BHA, p-hydroxyanisole, BHT, t-butyl hydroquinone, caffeic acid, Camellia sinensis Oil, chitosan ascorbate, chitosan glycolate, chitosan salicylate, chlorogenic acids, cysteine, cysteine HCl, decyl mercaptomethylimidazole, erythorbic acid, diamylhydroquinone, di-t-butylhydroquinone, dicetyl thiodipropionate, dicyclopentadiene/t-butylcresol copolymer, digalloyl trioleate, dilauryl thiodipropionate, dimyristyl thiodipropionate, dioleyl tocopheryl methylsilanol, isoquercitrin, diosmine, disodium ascorbyl sulfate, disodium rutinyl disulfate, distearyl thiodipropionate, ditridecyl thiodipropionate, dodecyl gallate, ethyl ferulate, ferulic acid, hydroquinone, hydroxylamine HCl, hydroxylamine sulfate, isooctyl thioglycolate, kojic acid, madecassicoside, magnesium ascorbate, magnesium ascorbyl phosphate, melatonin, methoxy-PEG-7 rutinyl succinate, methylene di-t-butylcresol, methylsilanol ascorbate, nordihydroguaiaretic acid, octyl gallate, phenylthioglycolic acid, phloroglucinol, potassium ascorbyl tocopheryl phosphate, thiodiglycolamide, potassium sulfite, propyl gallate, rosmarinic acid, rutin, sodium ascorbate, sodium ascorbyl/cholesteryl phosphate, sodium bisulfite, sodium erythorbate, sodium metabisulfide, sodium sulfite, sodium thioglycolate, sorbityl furfural, tea tree (Melaleuca aftemifolia) oil, tocopheryl acetate, tetrahexyldecyl ascorbate, tetrahydrodiferuloylmethane, tocopheryl linoleate/oleate, thiodiglycol, tocopheryl succinate, thiodiglycolic acid, thioglycolic acid, thiolactic acid, thiosalicylic acid, thiotaurine, retinol, tocophereth-5, tocophereth-10, tocophereth-12, tocophereth-18, tocophereth-50, tocopherol, tocophersolan, tocopheryl linoleate, tocopheryl nicotinate, tocoquinone, o-tolyl biguanide, tris(nonylphenyl) phosphite, ubiquinone, and zinc dibutyldithiocarbamate.

Some examples of oxidizing agents are ammonium persulfate, calcium peroxide, hydrogen peroxide, magnesium peroxide, melamine peroxide, potassium bromate, potassium caroate, potassium chlorate, potassium persulfate, sodium bromate, sodium carbonate peroxide, sodium chlorate, sodium iodate, sodium perborate, sodium persulfate, strontium dioxide, strontium peroxide, urea peroxide, and zinc peroxide.

Some examples of reducing agents are ammonium bisufite, ammonium sulfite, ammonium thioglycolate, ammonium thiolactate, cystemaine HCl, cystein, cysteine HCl, ethanolamine thioglycolate, glutathione, glyceryl thioglycolate, glyceryl thioproprionate, hydroquinone, p-hydroxyanisole, isooctyl thioglycolate, magnesium thioglycolate, mercaptopropionic acid, potassium metabisulfite, potassium sulfite, potassium thioglycolate, sodium bisulfite, sodium hydrosulfite, sodium hydroxymethane sulfonate, sodium metabisulfite, sodium sulfite, sodium thioglycolate, strontium thioglycolate, superoxide dismutase, thioglycerin, thioglycolic acid, thiolactic acid, thiosalicylic acid, and zinc formaldehyde sulfoxylate.

An example of a skin bleaching agent is hydroquinone.

Some examples of skin protectants are allantoin, aluminium acetate, aluminium hydroxide, aluminium sulfate, calamine, cocoa butter, cod liver oil, colloidal oatmeal, dimethicone, glycerin, kaolin, lanolin, mineral oil, petrolatum, shark liver oil, sodium bicarbonate, talc, witch hazel, zinc acetate, zinc carbonate, and zinc oxide.

The formulations of the present invention also include diluents. Such diluents are often necessary to decrease the viscosity of the formulation sufficiently for application.

Examples of diluents include silicon containing diluents such as hexamethyldisiloxane, octamethyltrisiloxane, and other short chain linear siloxanes, cyclic siloxanes such as octamethylcyclotetrasiloxane and decamethylcyclopentasiloxane, dodecamethylcyclohexasiloxane, organic diluents such as butyl acetate, alkanes, alcohols, ketones, esters, hydrofluorocarbons or any other material which can dilute the formulation without adversely affecting any of the component materials of the formulation or the curing time. On application, however, the diluent often substantially volatilises leaving the other component materials on the desired site.

Further materials suitable for personal care are well known to the person skilled in the art and are described in many text books as well as other publications.

Specific personal care compositions include skin care compositions, hair care compositions and nail care compositions. Skin care compositions include shower gels, soaps, hydrogels, creams, lotions, balms, foundations, lipsticks, eyeliners and blushes and may be in the form of water-in-oil emulsion, oil-in-water emulsion, water-in-silicone emulsion, silicone-in-water emulsion or multiple emulsions such as water-in-oil-in-water or oil-in-water-in-oil, or they may be anhydrous. Hair care compositions include shampoos, rinse off conditioners, leave-in conditioners, gels, pomades, cuticle coats, serum, sprays, colouring products and mascaras. Where hair care compositions are under the form of emulsions, they may be water-in-oil emulsion, oil-in-water emulsion, water-in-silicone emulsion, silicone-in-water emulsion. Hair care compositions may also be anhydrous. Nail care compositions include color coats, base coats, nail hardeners.

The amount of silicone acrylate hybrid composition present in compositions will be determined by the particular benefit to be obtained, for example, conditioning or care of the hair or skin. The particular level appropriate in different compositions according to the present invention is influenced by the particular composition into which it is formulated.

The general level of silicone acrylate hybrid composition in the personal care compositions may vary from 0.01% to 20% by weight, alternatively from 0.05% to 10%, alternatively from 0.1% to 5%.

In the personal care compositions, at least one personal care ingredient is present from 0.01% to 99.9% by weight of the final composition. The exact amount of each personal care ingredient in the final composition, in addition to the silicone acrylate hybrid composition, is a function of its nature and its purpose and is easily determined by the man skilled in the art. For example, a skin care lotion may comprise from 5% to 95% wt of emollient, from 0,01% to 5% wt ethylhexylmethoxy cinnamate, from 0,001% to 2% wt vitamin, from 10% to 90% wt of water and from 0.5% to 5% wt emulsifier, based on the weight of the final composition. A hair conditioner such as a rinse off may comprise from 0,001% to 5% wt cationic guar derivative, from 0.01% to 10% wt silicone oil, from 0.1% to 5% wt fatty alcohol and from 50% to 95% wt water based on the weight of the final composition. Generally, 0.01%-5% wt preservative is suggested in personal care compositions. An anhydrous cuticle coat may comprise from 0.5% to 99.5% wt silicone oil and from 0.01 to 0.1% wt fragrance based on the weight of the final composition. A lipstick may comprise from 5% to 50% wt waxes such as ozokerite wax, carnauba wax, from 5% to 50% wt oil phase, such as octyldodecanol, cyclomethicone, lanolin oil, shea butter, polypropylsilsesquioxane and from 1% to 25% wt of iron oxides and nacres based on the weight of the final composition. Several actives such as vitamins or peptides may be used.

The compositions are prepared by mixing the silicone acrylate hybrid composition with other compatible oil based ingredients to form an oil phase of the composition, potentially heating. The composition may be anhydrous or may be processed as an emulsion.

When making an emulsion composed of an oil phase and a water phase, the emulsifiers may be added to the appropriate phase, and the oil and aqueous phases may then mixed together to form the final composition. Anhydrous emulsions of oil phases with non aqueous phases such as glycols may also be prepared.

When the silicone acrylate hybrid composition is in the form of an emulsion, it may be mixed with the ingredients of the aqueous phase and be processed further as appropriate.

The composition may be adjusted for pH. Sensitive ingredients may further be added as appropriate, such as fragrances, nacres. Mixing devices are those generally used by the man skilled in the art to prepare personal care compositions and include mixing vessels with paddles, stirrers, homogenisers, scrapers and other equipment which is known to the person skilled in the art. The compositions may be prepared at temperatures ranging from 15° C. to 90° C., alternatively from 20° C. to 60° C., or alternatively at room temperature (20° C.-25° C.).

The compositions generally generate a conditioning benefit of the substrate, skin, mucosa, hair, or nail. Benefits obtained from using a hair care compositions include one or more of the following benefits: hair conditioning, softness, detangling ease, hold, color lasting, shine. Benefits obtained from using the skin care compositions according to the invention include one or more of the following benefits: skin softness, suppleness, wash off resistance, make up durability. Benefits obtained from using the nail care compositions according to the invention include one or more of the following benefits: shock and/or scratch resistance, crack absence, long lasting color.

### EXAMPLES

The following examples are included to demonstrate embodiments of the invention.

It should be appreciated by those of skill in the art that the techniques disclosed in the examples which follow represent techniques discovered by the inventor to function well in the practice of the invention, and thus may be considered to constitute typical modes for its practice. However, those of skill in the art should, in light of the present disclosure, appreciate that many changes may be made in the specific embodiments which are disclosed and still obtain a like or similar result without departing from the spirit and scope of the invention. All percentages are in wt. %. Stability is measured using Brookfield rheometer at weekly and monthly intervals, up to 12 months, at room temperature (20° C.-25° C.). Stability is defined as a constant viscosity over a certain time.

### Examples 1 to 3 and Comparative Examples 1 to 5

The components used in the following examples are as follows:
EAS is a silicon-containing pressure sensitive adhesive composition and is 63.4% weight solids in ethyl acetate. EAS is produced through a condensation reaction of a silanol endblocked polydimethylsiloxane (PDMS) with a silicate resin and that is endblocked with a silicon-containing capping agent which provides the acrylate or methacrylate functionality.
2-EHA is 2-ethylhexyl acrylate commercially available from Aldrich.
MA is methyl acrylate commercially available from Aldrich.
MMA is methyl methacrylate commercially available from Aldrich.
BA is butyl acrylate commercially available from Aldrich.
Vazo® 67 is 2,2′-azobis(methylbutyronitrile) commercially available from Dupont.

The glass transition temperatures (Tg) of a particular Example were determined by Differential Scanning Calorimetry (DSC). Approximately 2 to 4 milligrams of dried material was loaded into a standard aluminum pan. The aluminum pan with sample was placed into the cell of a DSC. The sample was first cooled to <−150° C. and then heated to +150° C. at a rate of 10° C./minute under a helium purge. The individual Tg is reported as the half-height of the material transition. One material may have multiple thermal transitions based on its composition.

### Example 1

109.25 g of MMA, 100.78 g of BA, 331.00 g of EAS, 50.81 g of ethyl acetate solvent and 0.305 g Vazo® 67 were added to form a pre-reaction mixture. The pre-reaction mixture was allowed to stir 15 minutes until thoroughly homogeneous. After mixing, 147.00 g of the pre-reaction mixture and 410.00 g of ethyl acetate solvent were added to a reactor. The remaining portion of the pre-reaction mixture was added to a separate reservoir. Heating and mixing was then begun on the mixture in the reactor. The reaction temperature was set at 78° C. As soon as the reaction temperature was achieved, the mixture was allowed to react for 30 minutes prior to adding more of the pre-reaction mixture to the reactor. Once 30 minutes elapsed, the mixture in the reservoir was then added at a rate of 1.85 grams/minutes for 240 minutes until the mixture in the reservoir was finished. The mixture in the reactor was then reacted at 78° C. for an additional 395 minutes to form the silicone acrylate hybrid composition. Upon completion, the silicone acrylate hybrid composition was allowed to cool to room temperature before being removed from the reactor. The silicone acrylate hybrid composition was then dried in a forced air oven at 150° C. for 60 minutes to remove the ethyl acetate solvent. The final product was opaque in color. Example 1 is characterized by a Tg(1) of −119° C. and a Tg(2) of 46° C.

### Example 2

149.12 g of MMA, 61.08 g of BA, 311.07 g of EAS, 50.94 g of ethyl acetate solvent and 0.305 g Vazo® 67 were added to form a pre-reaction mixture. The pre-reaction mixture was allowed to stir 15 minutes until thoroughly homogeneous. After mixing, 140.27 g of the pre-reaction mixture and 410.00 g of ethyl acetate solvent were added to a reactor. The remaining portion of the pre-reaction mixture was added to a separate reservoir. Heating and mixing was then begun on the mixture in the reactor. The reaction temperature was set at 78° C. As soon as the reaction temperature was achieved, the mixture was allowed to react for 30 minutes prior to adding more of the pre-reaction mixture to the reactor. Once 30 minutes elapsed, the mixture in the reservoir was then added at a rate of 2.16 grams/minutes for 200 minutes until the mixture in the reservoir was finished. The mixture in the reactor was then reacted at 78° C. for an additional 415 minutes to form the silicone acrylate hybrid composition. Upon completion, the silicone acrylate hybrid composition was allowed to cool to room temperature before being removed from the reactor. The silicone acrylate hybrid composition was then dried in a forced air oven at 150° C. for 60 minutes to remove the ethyl acetate solvent. The final product was opaque in color. Example 2 is characterized by a Tg(1) of −119° C. and a Tg(2) of 79° C.

### Example 3

180.58 g of MMA, 29.35 g of BA, 332.02 g of EAS, 52.45 g of ethyl acetate solvent and 0.305 g Vazo® 67 were added to form a pre-reaction mixture. The pre-reaction mixture was allowed to stir 15 minutes until thoroughly homogeneous. After mixing, 134.43 g of the pre-reaction mixture and 409.00 g of ethyl acetate solvent were added to a reactor. The remaining portion of the pre-reaction mixture was added to a separate reservoir. Heating and mixing was then begun on the mixture in the reactor. The reaction temperature was set at 78° C. As soon as the reaction temperature was achieved, the mixture was allowed to react for 30 minutes prior to adding more of the pre-reaction mixture to the reactor. Once 30 minutes elapsed, the mixture in the reservoir was then added at a rate of 2.71 grams/minutes for 170 minutes until the mixture in the reservoir was finished. The mixture in the reactor was then reacted at 78° C. for an additional 560 minutes to form the silicone acrylate hybrid composition. Upon completion, the silicone acrylate hybrid composition was allowed to cool to room temperature before being removed from the reactor. The silicone acrylate hybrid composition was then dried in a forced air oven at 150° C. for 60 minutes to remove the ethyl acetate solvent. The final product was opaque in color. Example 3 is characterized by a Tg(1) of −116° C. and a Tg(2) of 102° C.

### Comparative Example 1

PSA 1, is a conventional, i.e., uncapped, silicone PSA that is produced through the condensation reaction between a hydroxy end-blocked polydimethylsiloxane (PDMS) with a viscosity of 50,000 cP and a hydroxy end-blocked silicate resin at a compositional ratio of 35% PDMS to 65% resin.

### Comparative Example 2

PSA 2, is a conventional, i.e., uncapped, silicone PSA that is produced through the condensation reaction between a hydroxy end-blocked polydimethylsiloxane (PDMS) with a viscosity of 13,500 cP and a hydroxy end-blocked silicate resin at a compositional ratio of 35% PDMS to 65% resin.

### Comparative Example 3

PSA 3, is a conventional, i.e., uncapped, silicone PSA that is produced through the condensation reaction between a hydroxy end-blocked polydimethylsiloxane (PDMS) with a viscosity of 13,500 cP and a hydroxy end-blocked silicate resin at a compositional ratio of 40% PDMS to 60% resin.

### Comparative Example 4

PSA 4, is a silicone acrylate hybrid PSA that is produced through a radical polymerization between a silicon-containing PSA, 2-ethylhexyl acrylate and methyl acrylate. Comparative Example 4 is characterized by a Tg(1) of −123° C. and a Tg(2) of −32° C.

### Comparative Example 5

176.02 g of 2-EHA, 96.87 g of MA and 0.395 g Vazo® 67 were added to form a pre-reaction mixture. The pre-reaction mixture was allowed to stir 15 minutes until thoroughly homogeneous. After mixing, 70.47 g of the pre-reaction mixture and 375.67 g of ethyl acetate solvent were added to a reactor. The remaining portion of the pre-reaction mixture was added to a separate reservoir. Heating and mixing was then begun on the mixture in the reactor. The reaction temperature was set at 78° C. As soon as the reaction temperature was achieved, the mixture was allowed to react for 60 minutes prior to adding more of the pre-reaction mixture to the reactor. Once 60 minutes elapsed, the mixture in the reservoir was then added at a rate of 1.22 grams/minutes for 166 minutes until the mixture in the reservoir was finished. The mixture in the reactor was then reacted at 78° C. for an additional 570 minutes to form the acrylate PSA composition. Upon completion, the silicone acrylate hybrid composition was allowed to cool to room temperature before being removed from the reactor. The silicone acrylate hybrid composition was then dried in a forced air oven at 150° C. for 60 minutes to remove the ethyl acetate solvent. The final product was clear in color. Comparative Example 5 is characterized by a Tg(1) of −40° C.

### Example 4

Substantivity on skin: a film of material is formed over an ink spot previously made on the skin, and the test area is then washed with a sodium laureth sulfate dilution at 9%.

The resistance of the ink spot is evaluated visually by panellists. In the present test, Examples 1, 2 and 3, under the form of an individual 40 wt % dilution, have been proven surfactant resistant films, resisting to several washes, and having to be removed using a cosmetic solvent such as cyclomethicone.

### Examples 5 to 7 and Comparative Example 6

Homogeneous oil-in-water cream compositions were prepared from the components in Table 1 using the following procedure:
Mix ingredients of phase A
Slowly add ingredients of phase A (drop by drop) into phase B at 1200 rpm
Add phase C with mixing (1200 rpm)
Pass through Silverson (1min/100g—Speed: 5000 rpm)

TABLE 1

| | Compar- | | | |
|---|---|---|---|---|
| | ative | Exam- | Exam- | Exam- |
| Ingredients (% wt) | Example 6 | ple 5 | ple 6 | ple 7 |
| Phase A | | | | |
| Dimethicone 5 cSt | 20 | 10 | 10 | 10 |
| Example 1 dispersed in | | 10 | | |
| dimethicone 1.5 cSt (50/50) | | | | |
| Example 2 dispersed in | | | 10 | |
| dimethicone 1.5 cSt (50/50) | | | | |
| Example 3 dispersed in | | | | 10 |
| dimethicone 1.5 cSt (50/50) | | | | |
| Phase B | | | | |
| Water | 78 | 78 | 78 | 78 |
| Phase C | | | | |
| Polyacrylamide (and) C13-14 | 2 | 2 | 2 | 2 |
| isoparaffin (and) Laureth-7 | | | | |

### Example 8 and Comparative Examples 7 and 8

Homogeneous water-in-silicone cream compositions were prepared from the components in Table 2 using the following procedure:
Mix ingredients of phase A
Mix ingredients of phase B
Slowly add ingredients of phase B (drop by drop) into phase A at 700 rpm
Pass through Silverson (1min/100g—Speed: 5000 rpm)

TABLE 2

| | Compar- | Compar- | |
|---|---|---|---|
| | ative | ative | Exam- |
| Ingredients (% wt) | Example 7 | Example 8 | ple 8 |
| Phase A | | | |
| Cyclopentasiloxane (and) | 10 | 10 | 10 |
| PEG/PPG-18/18 dimethicone | | | |
| Cyclopentasiloxane | 15 | 3 | 5 |
| Isododecane (and) | | 12 | |
| Acrylates/Polytrimethylsiloxy- | | | |
| methacrylate Copolymer | | | |
| Example 1 dispersed in | | | 10 |
| dimethicone 1.5 cSt (50/50) | | | |
| Example 2 dispersed in | | | |
| dimethicone 1.5 cSt (50/50) | | | |
| Example 3 dispersed in | | | |
| dimethicone 1.5 cSt (50/50) | | | |
| PPG-3 Myristyl Ether | 0.5 | 0.5 | 0.5 |
| Phase B | | | |
| Water | 72.5 | 72.5 | 72.5 |
| Sodium chloride | 2 | 2 | 2 |

### Example 9

A homogeneous water-in-oil sun cream composition was prepared from the components in Table 3 using the following procedure:
Mix phase A ingredients and heat to 60° C.
Mix phase B ingredients
Add phase B very slowly into phase A under very strong agitation
When addition is complete, leave under agitation for an additional 5 minutes

TABLE 3

| Ingredients (% wt) | Example 9 |
|---|---|
| Phase A | |
| Octyl methoxycinnamate | 4 |
| C12-15 alkyl benzoate | 7 |
| Lauryl PEG/PPG-18/18 methicone | 2 |
| Cyclomethicone | 5 |
| Example 1 dispersed in dimethicone | 5 |
| 1.5 cSt (50/50) | |
| Phase B | |
| Propylene glycol | 3.5 |
| Water | 72.5 |
| Sodium chloride | 1 |

### Examples 10 to 12 and Comparative Example 9

Homogenous foundation compositions were prepared from the components in Tables 4 and 5.

Procedure to prepare Foundation compositions:

1) First, the pigment premix is prepared, as described in Table 4:
Mix all the pigments together
Put the cyclomethicone in final beaker, and gradually add the pigments, mix using a high shear mixer (ultra Turrax)
Mix with Ultra Turrax before putting into oil phase of foundation, in order to re-suspend the pigments
   2) Then the foundation compositions are prepared, as described in Table 5:
Combine ingredients in phase A, mix until uniform using a dual blade, turbulent style mixing action
Combine the ingredients in phase B in separate beaker, mix until uniform
Increase mixing speed of phase A to about 1400 rpm and very slowly add phase B
Continue mixing for an additional 10 minutes

TABLE 4

| | Pigment |
|---|---|
| Ingredients (% wt) | premix |
| Cyclomethicone | 50 |
| Titanium dioxide | 13.16 |
| red Iron oxide | 11.41 |
| Yellow iron oxide | 18.26 |
| Black iron oxide | 7.17 |

TABLE 5

| | | | | Compar- |
|---|---|---|---|---|
| | Exam- | Exam- | Exam- | ative |
| Ingredients (% wt) | ple 10 | ple 11 | ple 12 | Example 9 |
| Phase A | | | | |
| Pigment premix | 19.12 | 19.12 | 19.12 | 19.12 |
| Cyclopentasiloxane and | 7.24 | 7.24 | 7.24 | 7.24 |
| PEG/PPG18/18 dimethicone | | | | |
| Example 1 dispersed in | 9.67 | — | — | — |
| dimethicone 1.5 cSt (50/50) | | | | |
| Example 2 dispersed in | — | 9.67 | — | — |
| dimethicone 1.5 cSt (50/50) | | | | |
| Example 3 dispersed in | — | — | 9.67 | — |
| dimethicone 1.5 cSt (50/50) | | | | |
| Isododecane (and) | — | — | — | 12.1 |
| Acrylates/Polytrimethylsiloxy- | | | | |
| methacrylate Copolymer | | | | |
| Isododecane | 9.67 | 9.67 | 9.67 | 7.3 |
| Phase B | | | | |
| Water | 53.1 | 53.1 | 53.1 | 53.1 |
| Sodium chloride | 1 | 1 | 1 | 1 |
| Polysorbate-20 | 0.2 | 0.2 | 0.2 | 0.2 |
| Color loss (%) between before | 1.29 | 1.58 | 1.40 | 1.47 |
| and after 20 insults | | | | |

Examples 10 to 12 and Comparative Example 9 were evaluated for their durability:
A film of foundation is applied on a collagen sheet, and let to dry
Color is measured—“before”
A rubbing device is passing over the collagen sheets
Color is measured after 20 insults on the film of foundation—“after”
Difference of color is calculated between “before” and “after” (Color loss in %)

Results indicate Examples 1, 2 and 3 provide as much foundation durability as the benchmark film former Acrylates/Polytrimethylsiloxymethacrylate Copolymer (in Comparative Example 9).

### Example 13 and Comparative Example 10

Examples 1, 2 and 3 were incorporated at 1wt % into a commercial nail varnish successfully, providing for Example 13, proving their compatibility with the different ingredients present.

The ingredients contained in the commercial nail varnish (NIVEA Beauté, Calcium Power, 34 Currant)—Comparative Example 10—are: Butyl Acetate, Ethyl Acetate, Nitrocellulose, Acetyl Tributyl Citrate, Isopropyl Alcohol, Adipic Acid/Neopentyl Glycol/Trimelitic Anhydride Copolymer, Stearalkonium Bentonite, Acrylates Copolymer, Calcium Pantothenate, Cetraria Islandica Extract, Chondrus Crispus, Styrene/Acrylates Copolymer, Trimethylpentanediyl Dibenzoate-1, Polyvinyl Butyral, N-Butyl Alcohol, Aqua, Propylene Glycol, Butylene Glycol, Mica, Silica, Tin Oxide, Calcium Borosilicate, Synthetic Fluorphlogopite, Polyethylene Terephtalate, and may also be: Cl: 15850, 15880, 19140, 2090, 47000, 60725, 74160, 77000, 77007, 77163, 77491, 77492, 77499, 77510, 77742, 77891.

### Examples 14 to 16 and Comparative Examples 11 to 13

Examples 1, 2 and 3 were incorporated at 3.75wt % into a commercial permanent hair colouring product successfully, providing for Examples 14, 15 and 16 respectively, proving their compatibility with the different ingredients present.

The ingredients contained in the commercial permanent hair colouring product (L'Oréal Paris, Féria Préférence, 6.60 Red Fiction) are: 1) for the color phase: Aqua, Trideceth-2 Carboxamide MEA, Propylene Glycol, Hexylene Glycol, PEG-2 Oleamine, Polyglyceryl-4 Oleyl Ether, Oleyl Alcohol, Alcohol Denat., Ammonium Hydroxide, Polyglyceryl-2 Oleyl Ether, Oleic Acid, Sodium Diethylaminopropyl Cocoaspartamide, 4-Amino-2-Hydroxytoluene, p-Aminophenol, Sodium Metabisulfite, Eugenol, 6-Hydroxyindole, Ammonium Acetate, 2-methyl-5-Hydroxyethylaminophenol, Pentasodium Pentetate, Linalool, p-Phenylenediamine, Alpha-isomethyl ionone, Resorcinol, Citronellol, Erythorbic Acid, Parfum and 2) for the developer phase: Aqua, Hydrogen Peroxide, Cetearyl Alcohol, Sodium Stannate, Trideceth-2-Carboxamide MEA, Pentasodium Pentetate, Phosphoric Acid, Ceteareth-25, Tetrasodium Pyrophosphate, Glycerin.

Procedure to prepare colouring product

1) Add active of product in developer part as described in Table 6:
Check the compatibility (using a glass bottle)
   2) Prepare hair colouring mixture to apply on hair:
Mix 3 g of the developer phase and 3 g of color phase
Apply directly on dry hair: 6 g of mix on 6 g hair
Lather for 30 seconds
Wrap in aluminium foil for 20 minutes
Rinse under tap water at 37° C. for 2 minutes
Comb tresses flat
Repeat in order to have 3 tresses per product
Let dry on aluminium foil for the night
   3) Assess color and color loss (colorimeter: ColorSphere BYK Gardner):
Measure color after colouring—before washes
Wash tresses 5 times with sodium laureth sulphate dispersion at 9% active
Measure color after the 5 washes
Calculate Delta E between before and after washes—color loss:

ΔE*=\sqrt{(L₁−L₂)²+(a₁−a₂)²+(b₁−b₂)²}

TABLE 6

| | Example | Example | Example | Comparative | Comparative | Comparative |
|---|---|---|---|---|---|---|
| Ingredients (% wt) | 14 | 15 | 16 | Example 11 | Example 12 | Example 13 |
| Phase A | | | | | | |
| Developer phase | 87.5 | 87.5 | 87.5 | 87.5 | 87.5 | 87.5 |
| Example 1 dispersed in dimethicone 1.5 | 7.5 | | | | | |
| cSt (50/50) | | | | | | |
| Example 2 dispersed in dimethicone 1.5 | | 7.5 | | | | |
| cSt (50/50) | | | | | | |
| Example 3 dispersed in dimethicone 1.5 | | | 7.5 | | | |
| cSt (50/50) | | | | | | |
| Cyclopentasiloxane (and) | | | | | | 12.5 |
| Acrylates/Polytrimethylsiloxymethacrylate | | | | | | |
| Copolymer | | | | | | |
| Cyclomethicone (and) | | | | | 7.5 | |
| Trimehtylsiloxysilicate | | | | | | |
| Dimethicone 1.5 cSt | 5 | 5 | 5 | 12.5 | 5 | |

Results of color loss (delta E) in Table 7 indicate that Comparative Example 11 containing only dimethicone has lost the most of color, compared to the other samples. Example 2 composition, in Example 15, offers the best color protection compared to the other silicone acrylate hybrid compositions in Examples 14 and 16, which are however satisfying in regard with Comparative Examples 12 and 13, usually materials recommended for color protection (blend of Trimehtylsiloxysilicate and blend of Acrylates/Polytrimethylsiloxymethacrylate Copolymer respectively).

Hair tresses treated with Example 16 and Comparative Example 13 are very easy to comb, slightly better than hair tresses treated with Example 14 or 15, but definitely easier than Comparative Example 12. Hair tresses treated with Comparative Example 11 have a greasy look and feel. Hair tresses treated with Examples 14, 15, 16 and Comparative Example 13 are very soft, softer than hair tresses treated with Comparative Example 12.

TABLE 7

| Color | | | | Compar- | Compar- | Compar- |
|---|---|---|---|---|---|---|
| loss | | | | ative | ative | ative |
| of hair | Example | Example | Example | Example | Example | Example |
| tresses | 14 | 15 | 16 | 11 | 12 | 13 |
| Delta E | 3.76 | 2.83 | 4.63 | 5.87 | 4.85 | 3.51 |
| between | | | | | | |
| before | | | | | | |
| and after | | | | | | |
| 5 washes | | | | | | |

### Example 17

Example 1 was incorporated in a lipstick, as disclosed in Table 8, providing for

Example 17. The lipstick has a good pay off, shows long lasting properties and is comfortable to wear.

Procedure to prepare the lipstick:
Mix ingredients of phase A together
Heat phase A to 85° C. mixing until homogeneous
Cool to 75° C. and add phase B mixing until homogeneous
Pour into lipstick mold and leave to set at 0° C.

TABLE 8

| Ingredients (% wt) | Example 17 |
|---|---|
| Phase A | |
| Synthetic Wax (and) BHT | 10 |
| Polydecene | 62 |
| Synthetic Wax | 8 |
| Mica (and) CI 77891 (and) CI 75470 | 15 |
| Phase B | |
| Example 1 dispersed in dimethicone | 5 |
| 1.5 cSt (50/50) | |

1. A composition comprising (A) a silicone acrylate hybrid composition, and (B) at least one personal care ingredient.

2. The composition of claim 1 where the silicone acrylate hybrid composition (A) comprises the reaction product of:
I. a silicon-containing pressure sensitive adhesive composition comprising acrylate or methacrylate functionality
II. an ethylenically unsaturated monomer selected from the group of aliphatic acrylates, aliphatic methacrylates, cycloaliphatic acrylates, cycloaliphatic methacrylates, and combinations thereof, each of said compounds having up to 20 carbon atoms in the alkyl radical; and
III. an initiator.

3. The composition as claimed in claim 1 where the silicone acrylate hybrid composition (A) is present from 0.01% to 20% by weight and the at least one personal care ingredient (B) is present from 0.01 to 99.9% by weight.

4. The composition as claimed in claim 1 wherein the personal care ingredient is a skin care ingredient.

5. The composition as claimed in claim 4 wherein the skin care ingredient is selected from emollients, moisturizers, colorants, dyes, UV absorbers and sunscreens, antiperspirants, antioxidants, fragrances, antimicrobial or antibacterial or antifungal agents, pigments, preservatives, pH controlling agents, electrolytes, chelating agents, vegetable or botanical extracts, sebum absorbents or sebum control agents, vitamins and/or moisturizers, waxes, surface active materials such as surfactants or detergents or emulsifiers, thickeners, antioxidants and a cosmetically acceptable medium, such as water or mixtures thereof.

6. A composition as claimed in claim 4, which is a skin care composition.

7. A skin care composition as claimed in claim 6, which is a shower gel, soap, hydrogel, cream, lotion, balm, foundation, lipstick, eyeliner or blush.

8. The composition as claimed in claim 1 wherein the personal care ingredient is a hair care ingredient.

9. The composition as claimed in claim 8 wherein the hair care ingredient is selected from colorants, dyes, UV absorbers, preservatives, vegetable extracts, fatty alcohols, vitamins, fragrance, anti-dandruff agents, colour care additives, pearlising agents, pH controlling agents, electrolytes, chelating agents, styling agents, ceramides, amino-acid derivatives, suspending agents, surface active materials such as surfactants or detergents, thickeners, oxidizing agents, reducing agents and a cosmetically acceptable medium, such as water or mixtures thereof.

10. A composition as claimed in claim 8 which is a hair care composition.

11. Composition as claimed in claim 1, which is a shampoo, rinse off conditioner, leave-in conditioner, gel, pomade, cuticle coat, serum, spray, colouring product or mascara.

12. A method of treating a hair or skin substrate by applying to it the composition as claimed in claim 1.
